(19) United States
(12) Patent Application Publication
(10) Pub. No.: US 2014/0147908 A1
(43) Pub. Date: May 29, 2014

(54) METHOD FOR SPLITTING DROPLETS ON DEMAND IN MICROFLUIDIC JUNCTION

(71) Applicants: Slawomir Jakiela, Krosno (PL); Tomasz Kaminski, Wilkolaz (PL); Piotr Garstecki, Warszawa (PL)

(72) Inventors: Slawomir Jakiela, Krosno (PL); Tomasz Kaminski, Wilkolaz (PL); Piotr Garstecki, Warszawa (PL)

(73) Assignees: PZ CORMAY S.A., Lomianki (PL); INSTYTUT CHEMII FIZYCZNEJ POLSKIEJ AKADEMII NAUK, Warszawa (PL)

(21) Appl. No.: 14/235,448

(22) PCT Filed: Jul. 25, 2012
(86) PCT No.: PCT/EP2012/064640

§ 371 Date: Jan. 27, 2014

(30) Foreign Application Priority Data
Jul. 27, 2011 (PL) P-395776

(51) Int. Cl. G01N 1/28; C12N 1/20

(52) U.S. Cl. CPC G01N 1/28; C12N 1/20
USPC 435/252.8; 435/29; 137/2

(57) ABSTRACT

The invention relates to a method for splitting droplets on demand in a microfluidic junction, comprising the supply channel, the first drain channel and the second drain channel, the method comprises the following steps:
a. delivering a droplet (1) to the said microfluidic junction (3) through said supply channel (2) by means of a flow of continuous liquid through the supply channel (2) and said first drain channel,
b. stopping the flow in said first drain channel and opening the flow in said second drain channel until a fraction (7) of the droplet (1) is present in the second drain channel,
c. resuming the flow in the first drain channel and closing the flow in the second drain channel, at least until the fraction (7) of the said droplet (1) being present in the first drain channel separates from the rest of the droplet.

This application is a national phase application under 35 U.S.C. §371 of International Application Serial No. PCT/EP2012/064640, filed on Jul. 25, 2012, and claims the priority under 35 U.S.C. §119 to Polish Patent Application No. P-395776, filed on Jul. 27, 2011, which are hereby expressly incorporated by reference in their entirety for all purposes.

### TECHNICAL FIELD

The subject matter of the invention are technical solutions related to splitting of droplets on demand in a microfluidic junction, i.e., a method for splitting a droplet with known composition into two droplets in adjustable proportions, and automated techniques for splitting droplets on demand in such a microfluidic system.

### BACKGROUND OF THE INVENTION

The solutions according to the present invention, in combination with modules described in earlier patent applications of Prof Piotr Garstecki's research team (Polish patent applications No. P-390250, P-390251, P-393619 not published yet, and an international patent application No. PCT/PL2011/050002) may be used for a long-term culture of microorganisms or maintaining cell cultures, or for chemical analyses inside droplets and for detecting the outcome of these reactions as a function of controlled chemical composition of fluid samples and their positions inside the microfluidic systems. Particularly preferred systems according to the invention may be used for long-term studies of growth of microorganisms under various culture conditions, i.e., at varying medium concentration or presence of growth inhibitors, e.g., antibiotics and other substances affecting the physiology of microorganisms. A long-term culture relies on a cyclic removal of a fraction of a culture and feeding the culture with a strictly determined portion of fresh medium.

A growing number of reports on applications of microfluidic systems in biological sciences allow one to predict a rapid development of a lab-on-a-chip technology in near future. Particularly promising is the application of droplets generated in microchannels as miniaturised reactors, because of their small volume, from microliters, through nanoliters down to picoliters.

Typically, droplet-based microsystems possess a multitude of microfluidic channels, with their inlets and outlets that can join inside the system, where droplets containing solutions are surrounded by a non-miscible continuous phase. Further, the droplets inside the systems may be merged, transported along the channels while their contents are being mixed, stored under specific or varying conditions and finally sorted or split at channel junctions and recovered from the system. The use of microlaboratories to perform chemical and biochemical reactions inside microdroplets offers the following advantages [H. Song, D. L. Chen and R. F. Ismagilov, Ang. Chem. Int. Ed., 2006, 45, 7336-7356]: i) no dispersion of time of residence for fluid elements in a channel, ii) rapid mixing, iii) reaction kinetics can be easily controlled, iv) multiple reactions can be performed in parallel, v) low consumption of reagents, and vi) fast detection of the outcome of reactions (due to a low droplet volume the reaction products reach faster measurable concentrations).

These characteristics make the microdroplet-based microsystems a valuable tool for analytical chemistry, synthetic chemistry, biochemistry, microbiology, medical diagnostics or molecular diagnostics.

One of the indispensable operations performed inside the microfluidic droplet-based systems is droplet splitting. In the state of the art there are several methods for droplet splitting at a T-junction in a microfluidic system, wherein the continuous phase (i.e., the channel wall wetting phase) is a liquid [D. R. Link, S. L. Anna, D. A. Weitz, and H. A. Stone, Phys. Rev. Lett. 2004, 92, 4.] [J. Nie and R. T. Kennedy, Anal. Chem. 2010, 82, 7852-7856]. With an appropriate geometry it is possible to split a droplet in a ratio that is inversely proportional to flow resistances in each of the two branches of a T-junction. A drawback of this solution is, however, that the splitting ratio is predetermined by the geometry of the microfluidic system and the fact that the ratio cannot be changed in a controllable way during system operation.

In the state of the art there are many examples of studies and assays on cell cultures (bacteria, yeasts and mammalian cells) performed inside droplets in a microfluidic system [J. Q. Boedicker, L. Li, T. R. Kline, and R. F. Ismagilov, Lab Chip 2008, 8, 1265-1272] [C. H. J. Schmitz, A. C. Rowat, S. Köster, and D. A Weitz, Lab Chip 2009, 9, 44-49], [J. Clausell-Tormos, D. Lieber, J. C. Baret, A. El-Harrak, O. J. Miller, L. Frenz, J. Blouwolff, K. J. Humphry, S. Köster, H. Duan, C. Holtze, D. A. Weitz, A. D. Griffiths, and C. A. Merten, Chemistry & Biology 2008, 15, 427-437]. As a rule, however, the measurements performed in these systems have not included any analysis of cell physiology in a long period of time. The analysis in a long period of time requires keeping constant conditions for the cell growth, but the cultures of microbes show already after a few hours a deficiency of nutrients, the culture becomes saturated, and the growth is inhibited. Cyclic or continual removal of a fraction of a culture and feeding it with fresh nutrients allows for maintaining an uniform rate and reasonably identical conditions of growth.

In the state of the art there is a device—a chemostat [A. Novick and L. Szilard, Science 1950, 112, 715-716] that is capable of continuous substituting of a fraction of a culture with a fresh medium so that the substitution rate can be regulated. In the state of the art there are a few solutions allowing for chemostat miniaturisation: i) systems based on multi-layer elastomer systems [F. K. Balagadde, L. You, C. L. Hansen, F. H. Arnold, S. R. Quake, Science 2005, 309, 137], ii) miniaturised systems with a construction resembling chemostats used in the macro scale [N. Szita, P. Boccazzi, Z. Zhang, P. Boyle, A. J. Sinskey, K. F. Jensen, Lab Chip 2005, 5, 819], iii) systems making use of droplet movements on electrode surfaces, so called “digital microfluidics”[S. H. Au, S. C. C. Shih, A. R. Wheeler, Biomed. Microdevices 2011, 13, 41].

In the state of the art there is no method enabling and allowing for significant multiplication of the number of chemostats, while enabling to maintain a culture in each of them. In all the examples mentioned above, the multiplication of chemostats involves significant increase in the system complexity, and the number of key elements in the chip architecture increases in a linear proportion to the number of chemostats.

It is advisable to make use of two-phase flows, i.e., microdroplets travelling in microchannels with a diameter from a few to a few hundreds micrometers as a technology that is usable in long-term culture of microorganisms.

The inventors of the present invention noticed unexpectedly that it is possible to construct a microfluidic system allowing for a long-term culture and monitoring of microorganisms so that each droplet functions as an independent chemostat. Furthermore, it turned out unexpectedly that a system fabricated according to the invention allows to maintain a culture of microorganisms under varying conditions, in particular at varying concentration of substances affecting the growth of microorganisms, e.g., antibiotics, as well as at varying dilution rate (D), i.e., at varying rate of removal of saturated culture and feeding it with fresh culture medium.

### SUMMARY OF THE INVENTION

According to the invention, the method for splitting droplets on demand in a microfluidic junction, comprising the supply channel, the first drain channel and the second drain channel, is characterised in that it comprises the following stages:
a. supplying a droplet to the said microfluidic junction through the said supply channel by means of a flow of continuous liquid through this channel and the said first drain channel,
b. stopping the flow in the said first drain channel and opening the flow in the said second drain channel until a fraction of the said droplet is present in the said second drain channel,
c. resuming the flow in the said first drain channel and closing the flow in the said second drain channel, at least until the fraction of the said droplet being present in the said first drain channel separates from the rest of the droplet.

Preferably, the flows are controlled automatically, with a sensor, preferably a camera, located in the vicinity of the said microfluidic junction and connected directly or indirectly to the valves controlling the flows in the said supply channel, the said first drain channel and the said second drain channel, respectively.

In one of the preferred embodiments of the present invention, the said microfluidic junction is a T-junction, i.e., a junction wherein the said supply channel, the said first drain channel and the said second drain channel form with each other angles 180°, 90° and 90°, respectively.

In another preferred embodiment of the present invention, the said microfluidic junction is a Y-junction, i.e., a junction wherein the said supply channel, the said first drain channel and the said second drain channel form with each other angles 150°, 60° and 150°, respectively.

In yet another preferred embodiment of the present invention, the said microfluidic junction is a junction, wherein the said supply channel, the said first drain channel and the said second drain channel form with each other angles 120°, 120° and 120°, respectively.

Preferably, according to the invention, the said droplet is split in a volume ratio from 1:9 to 9:1, more preferably from 1:99 to 99:1, and most preferably from 1:999 to 999:1.

In one of the preferred embodiments of the invention, said droplet contains microorganisms, such as for example bacteria of E. coli culture, said droplet is split into two droplets and the method further comprises a step of
d. merging at least one of the newly formed droplets with a portion of a fresh nutrient for said microorganisms.

The portion of a fresh nutrient may further contain a substance affecting the growth of said microorganisms, such as for example chloramphenicol.

In such case, preferably, the method according to the invention further comprises a step of
e. re-circulating at least one of the newly formed droplets (8, 9) back and forth in a microfluidic channel, to incubate and monitor growth of said microorganisms.

In such embodiment, preferably, the steps d. and e. are repeated. In some applications, it is particularly preferred to repeat these steps in regular time intervals (with a period T) and with regular or irregular volume changes. In other applications, it is preferred to repeat these steps in irregular time intervals and with regular or irregular volume changes. Here the “volume changes” refer to volumes of the newly formed droplets, into which the initial droplet is split or to the volumes of portion of a fresh nutrient, which is added to at least one of the newly formed droplets. It should be understood that these volumes can be constant and repeatable in consecutive repetitions of steps d. and e. Or they may be changed regularly (for example—periodically) in consecutive repetitions of steps d. and e. Or they may be changed irregularly (for example—randomly) in consecutive repetitions of steps d. and e.

Said regular/irregular intervals and volume changes can be strictly correlated with monitored growth of said microorganisms on the basis of feedback to achieve, for example required growth rate of said microorganisms.

In the description below we present a method for splitting droplets on demand that is based on a controlled droplet positioning and control over the opening times of valves closing the outflow of the liquid from the branches of a T-junction. The T-junction is discussed here as a typical and non-limiting embodiment of the invention. For the purposes of present invention, the T-junction is defined as an intersection of three channels, with one of them supplying the fluids to the junction, and the remaining two draining off the fluids. The branches of the T-junction are aligned at 90°, 90° and 180° to each other. Competent persons will, however, easily notice that the methods presented here can be directly applied for any other angles.

The inventors of the present invention have noticed unexpectedly that it is possible to open the valve closing the outlet from one of the drain channels, at the time when the droplet being split is present in the T-junction, so that the droplet position and the duration of valve opening decide on the volumes of the newly created two droplets. In the state of the art there is a solution with a similar scheme of operation [Anal. Chem. 2008, 80, 6206-6213], the continuous phase in this solution is, however, a gas, and not a liquid (oil), which brings about many unfavourable consequences, including the following major ones: i) gases are compressible which makes controlling the droplets much more difficult and gaining a significant control over this process is not possible.

Moreover, in solutions known from the state of the art, the control over the flow of a larger number of droplets is not possible—it requires application of high pressures which generate significant fluctuations of gas volumes, as a result of their compressibility, and the entire system goes out of control; ii) water droplets that consist the continuous phase in the liquid-gas system are not entirely isolated from the system—they wet microchannel walls, which has an unfavourable effect in many applications—for instance increases the risk of cross-contamination between adjacent droplets; iii) in a system liquid (disperse phase)—gas (continuous phase), there occurs a problem related to evaporation of the liquid forming the droplets—the process changes the composition of the droplets, which no doubt is an unfavourable phenomenon.

The inventors of the present invention have noticed unexpectedly that it is possible to construct a microfluidic system allowing for splitting droplets on demand in a very broad and variable ratio, so that the ratio may be different for subsequent droplets that are split one after the other. The splitting ratio depends to some extent on the system geometry, it turned out unexpectedly, however, that it is possible to manipulate the volume ratio of two newly emerging droplets by appropriate droplet positioning and controlling the opening time of the valve closing the drain of a liquid from one of the branches of the T-junction. For instance, the maximum splitting ratio for a droplet with a volume of 1 μl, for a typical T-junction geometry (inlet channel with dimensions larger than 100×100 mm, and outlet channels with a diameter 100×100 μm, allow to split droplets at any volume ratio from 1:999 to 999:1).

### DETAILED DESCRIPTION OF DRAWINGS

Preferred embodiments are now explained with reference to the accompanying figures, wherein:

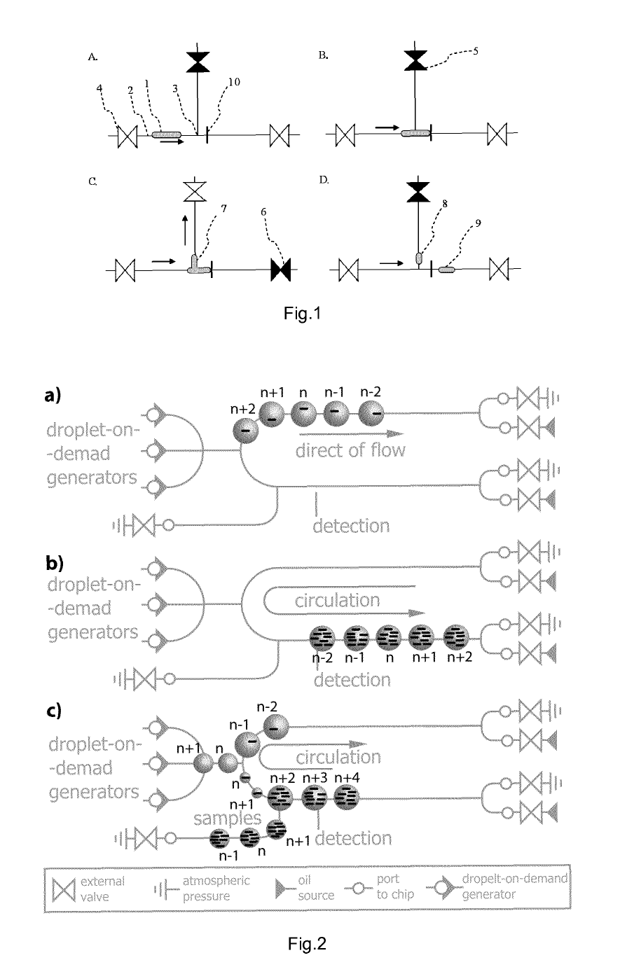

FIG. 1 shows a schematic diagram of a microsystem according to the invention that is used to split droplets on demand in a T-junction,

FIG. 2 shows a schematic diagram of a microsystem according to the invention that is used as a multiple chemostat inside the droplets,

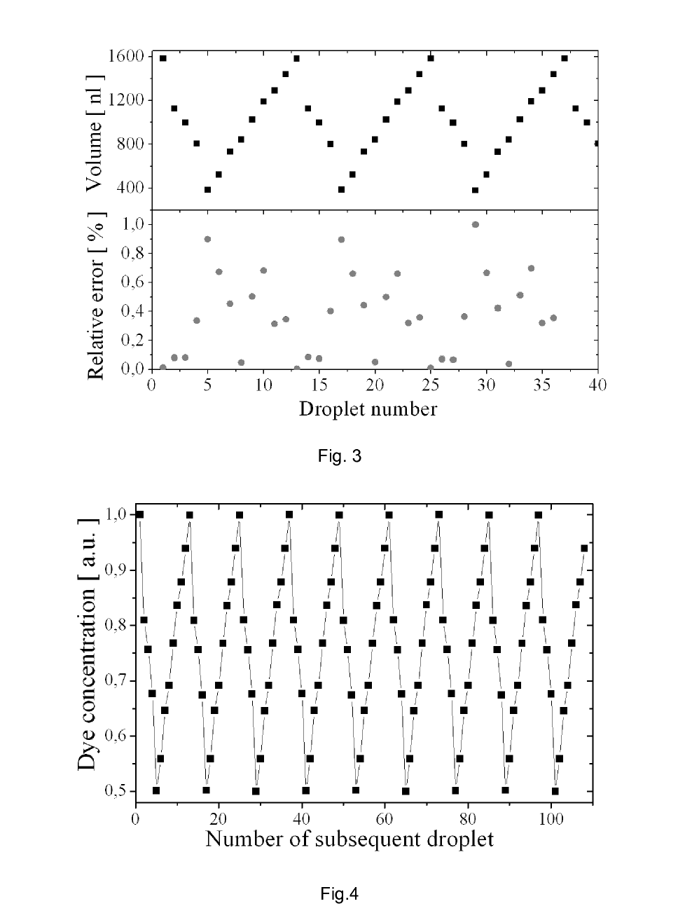

FIG. 3 shows a plot illustrating the droplet volume after asymmetrical splitting and the relative error of the droplet volume,

FIG. 4 shows a plot illustrating the change of dye concentration in a droplet resulting from droplet splitting and refilling the initial droplet volume with a specific fluid,

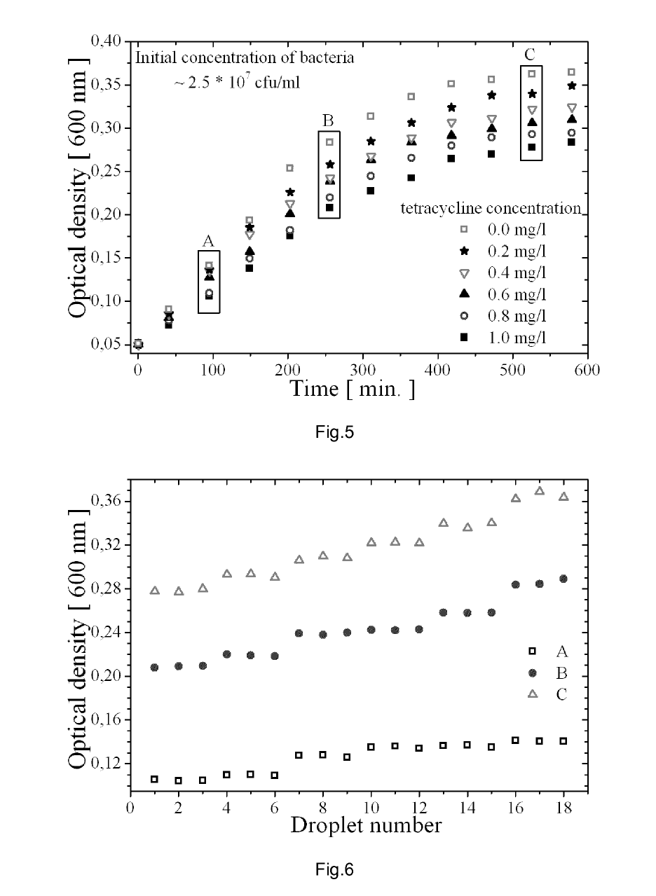

FIG. 5 shows a plot illustrating the increase of optical density in time in a multiple chemostat as a function of antibiotic concentration,

FIG. 6 shows a plot illustrating the increase of optical density in time in selected 9 microdroplets containing 3 different antibiotic concentrations,

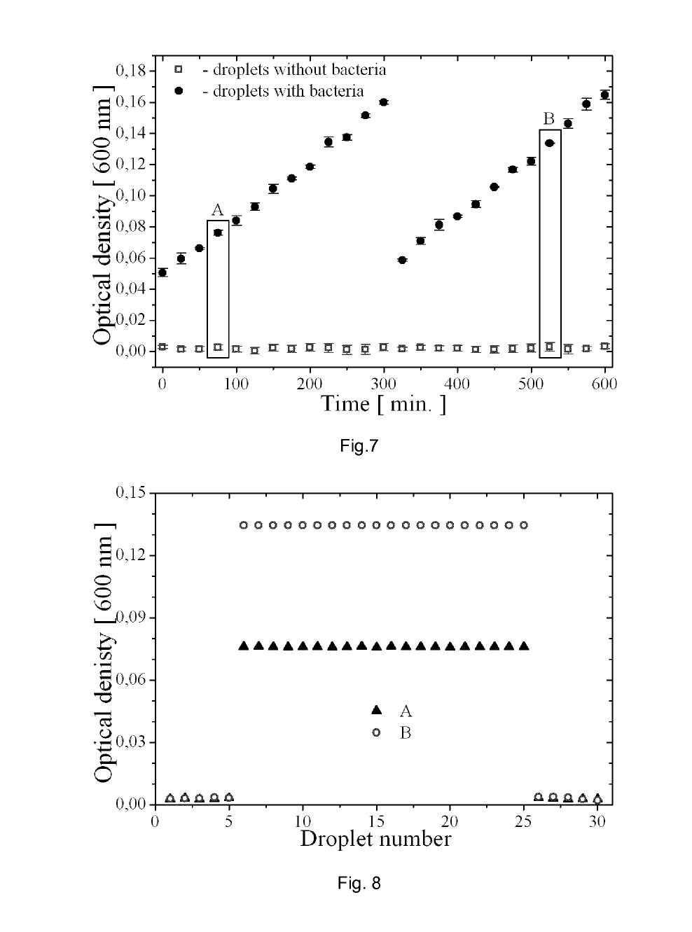

FIG. 7 shows a plot illustrating cyclic increase of optical density in time in a given droplet resulting from its splitting and refilling the initial droplet volume with a specific fluid, and the absence of an increase of optical density in a control droplet without bacteria, and

FIG. 8 shows a plot illustrating optical density for all droplets of a multiple chemostat at two selected measurement points.

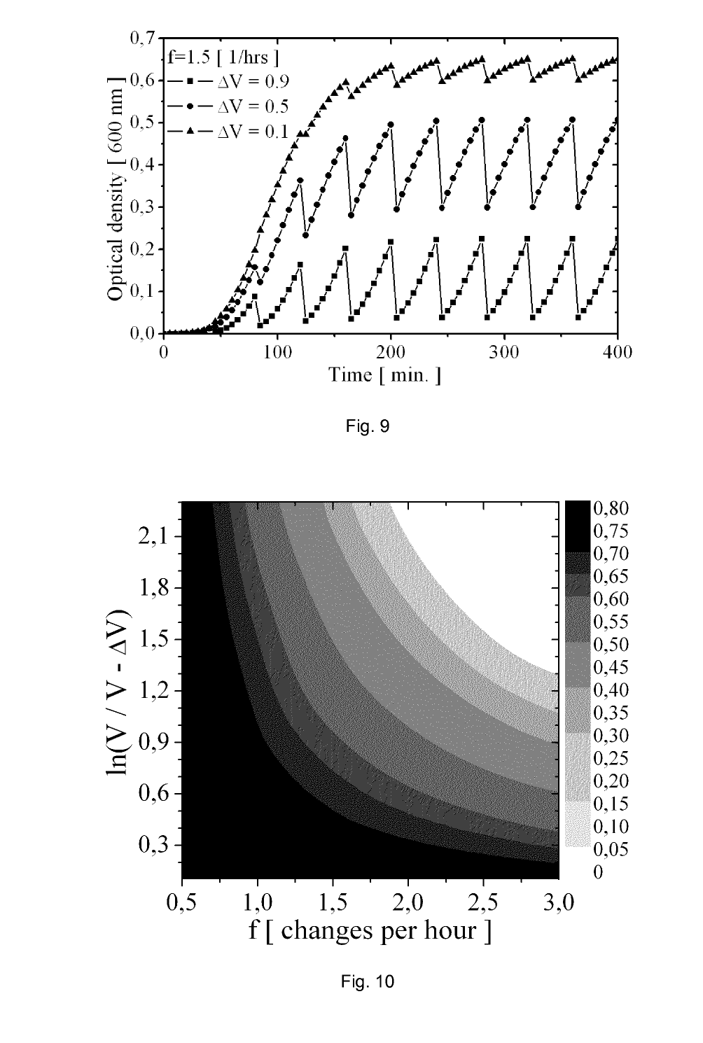

FIG. 9 shows a plot illustrating the cyclic change of optical density in a droplet containing E. Coli cultures in time for a fixed value of f and for three different values of ΔV.

FIG. 10 shows a map illustrating the maximum growth rate obtained for fixed values of f and ΔV. The gray bar (on the right ide) codes optical density (OD).

### BRIEF DESCRIPTION OF THE INVENTION

Embodiments of the present invention will be described hereinafter with reference to the accompanying drawings, in which the preferred exemplary embodiments of the invention are shown. The ensuing description is not intended to limit the scope, applicability or configuration of the disclosure. Rather, the ensuing description of the preferred exemplary embodiments will provide those skilled in the art with an enabling description for implementing the preferred exemplary embodiments of the disclosure. It should be noted that this invention may be embodied in different forms without departing from the spirit and scope of the invention as set forth in the appended claims.

### Example 1

In a preferred example of embodiment, a droplet residing in a microchannel is subject to splitting on demand in a T-junction. In the example illustrated schematically in FIG. 1A, the droplet 1 residing in channel 2 is displaced to the T-junction 3 by a stream of continuous liquid regulated by valve 4. In a preferred example, at the time when the droplet is residing in the T-junction (FIG. 1B), valve 5 that controls draining of fluids from one of the branches of the T-junction opens and at the same time valve 6 that controls draining from the other branch of the junction closes. This results in aspiration of a fraction of the volume of droplet 7 to the side channel of the T-junction. The duration of this operation is decisive for the droplet splitting ratio in the T-junction. Then, valves 5, 6 switch again, which results in droplet splitting into two droplets with fixed volumes 8, 9. In a preferred example, a sensor 10 is installed over or under the junction 3 to inform an electronic device (not shown in the Figure) about the flow of samples. In a preferred but non limiting embodiment it is an optical or electric sensor. In the example, the electronic device switches the valves 4, 5, 6 in such a way that it is possible to obtain different droplet splitting ratios. Preferably, a droplet being split has a length equal to a few channel widths—this may be attained by narrowing the channel in front of the T-junction.

### Example 2

In a preferred example of embodiment of a multiple chemostat inside droplets in a microfluidic system, a sequence of droplets 10 containing bacteria cultures is introduced into the system and re-circulated there and back (FIG. 2a) to incubate and monitor the growth of microorganisms 11. After a given period of time T, the droplets on demand 12 are split and a fraction of the culture 13 is removed (FIG. 2b). The splitting may be carried out with different splitting ratios, either set in advance or dependent on the results of measurement of microorganism concentration (measured as optical density, turbidimetry, luminescence or another marker of growth and life span of microorganisms). The frequency of splitting and the volume of the removed fraction are decisive for the dilution rate D:

$$D = \frac{ln ( 1 - F )}{T} \qquad ( 1 )$$

where D means the dilution rate, F means the fraction being substituted in subsequent dilutions, T is the time between subsequent dilutions. The next stage is to feed the droplets 14 with a portion of fresh culture medium 15 with a volume equal to that of the removed fraction of the culture 13 (FIG. 2c). Theoretically, the cycle may be repeated any number of times.

### Example 3

In a preferred example of embodiment of splitting droplets on demand it is possible to split droplets with a high accuracy, with the error not higher than 1%, whereas said error is lower for larger droplets. A typical plot presents the volume of a 2 μl droplet after splitting. It is characteristic that the splitting of the droplet does not deviate from the one preset by the operator, and, as mentioned above, the relative error between the demanded volume and that obtained is not greater than 1%.

### Example 4

In a preferred example of embodiment of splitting droplets on demand (FIG. 4) it is possible to split a droplet containing a dye, and subsequently to merge one of the newly formed droplets with a droplet that does not contain the dye, or with a droplet containing the dye so that the dye concentration may be increased or decreased in the same droplet, by very many splittings. A typical plot shows the change of dye concentration in a specific droplet (in this case 1 μl) as a result of droplet splitting and replenishment of initial droplet volume with a specific fluid. Due to both the favourable effect of droplet splitting and the method of droplet merging, the concentration inside the droplet changes essentially according to a predetermined scheme that is entirely related to the predetermined droplet splitting. Preferred repeatability of the presented phenomenon, with the relative error (between the concentration set by the operator of the microfluidic system and the concentration finally obtained) in the invention described here less than 1%, turns out to be of key importance, in particular for precise control of droplet composition.

### Example 5

In another, preferred example of the use of the invention, the system according to the invention may be used for a long-term culture of microorganisms (FIG. 5), including E. coli cultures, in presence of antibiotics or other substances affecting the growth of microorganisms. In the example, in a system analogous to the system shown in FIG. 2, long-term cultures of microorganisms were maintained with 6 different tetracycline concentrations, whereas each concentration was tested at least in three droplets used as microchemostats (FIG. 6). The growth of bacteria was monitored in equal time intervals with spectrophotometric measurements, using a light guide integrated with the system. The inventors have noticed unexpectedly that it is possible to determine the growth curves of bacteria for different antibiotic concentrations. Similarly unexpectedly, it turned out that these curves are highly reproducible.

### Example 6

Likewise, it is possible to use the same system to maintain a culture along with dilution of the culture (FIG. 7). In the presented example, after certain period of continuous growth, the droplet was split asymmetrically using the invention described in this application, which resulted in removal of 70% of the droplet volume, followed by merging the remaining 30% of the initial droplet with a droplet of fresh culture medium, with a volume equal to that of the removed culture fraction. In that way the bacteria gained new sources of nutrients and were able to continue their growth. The inventors of the present invention found unexpectedly that there was no unfavourable cross contamination between the droplets containing bacteria and the control droplets containing culture medium only, where no detectable growth of microorganisms took place (FIG. 8).

### Example 7

In a preferred example it is possible to split a droplet containing bacteria of E. coli culture (of volume V into two volumes: V−ΔV and ΔV), and subsequently to merge one of the newly formed droplet (of volume ΔV) with a droplet containing fresh nutrient (of volume V−ΔV). In the presented example, after certain period of continuous growth, the droplet was split asymmetrically (ΔV≠0.5 V) or symmetrically (ΔV=0.5 V) using the inventive method described in this application. And next newly formed droplet re-circulated back and forth on the chip to incubate and monitor the growth of microorganisms with the use of an in-line fibre optic spectrophotometer. After a given interval T, the described steps were iterated (repeated). The inventors noticed unexpectedly that for fixed values of T and ΔV the growth of bacteria is repetitive and the growth of the bacteria reached steady level prior to the next splitting after some cycles of splitting (FIG. 9). Therefore it is possible to plot the map of density of the colonies of bacteria expressed in the monitored value of optical density (OD) as a function of f=1/T in the unit of changes per hour and ΔV (FIG. 10).

While the principles of the disclosure have been described above in connection with specific examples and methods, it is to be clearly understood that this description is made only by way of example and not as limitation on the scope of the invention.

What is claimed is:

1-14. (canceled)

15. A method for splitting droplets on demand in a microfluidic junction, comprising a supply channel, a first drain channel and a second drain channel, the method comprising the steps of:
delivering a droplet to said microfluidic junction through the supply channel by means of a flow of continuous liquid through said supply channel and said first drain channel;
stopping the flow in said first drain channel and opening the flow in said second drain channel until a fraction of the droplet is present in the second drain channel;
resuming the flow in the first drain channel and closing the flow in the second drain channel, at least until the fraction of said droplet being present in the first drain channel separates from the rest of the droplet.

16. The method according to claim 15, wherein the flows are controlled automatically, with a sensor, preferably a camera, located in the vicinity of said microfluidic junction and connected directly or indirectly to three valves controlling the flows in said supply channel, said first drain channel and said second drain channel, respectively.

17. The method according to claim 15, wherein said microfluidic junction is a T-junction where the supply channel, the first drain channel, and the second drain channel form with each other angles of 180°, 90°, and 90°, respectively.

18. The method according to claim 15, wherein said microfluidic junction is a Y-junction where the supply channel, the first drain channel, and the second drain channel form with each other angles of 150°, 60° and 150°, respectively.

19. The method according to claim 15, wherein said microfluidic junction is a junction where the supply channel, the first drain channel, and the second drain channel form with each other angles of 120°, 120°, and 120°, respectively.

20. The method according to claim 15, wherein the droplet is split in a volume ratio from 1:9 to 9:1, preferably from 1:99 to 99:1, and most preferably from 1:999 to 999:1.

21. The method according to claim 15, wherein the droplet contains microorganisms such as bacteria of E. coli culture, and the droplet is split into two droplets.

22. The method according to claim 21 further comprises a step of merging at least one of the newly formed droplets with a portion of a fresh nutrient for said microorganisms.

23. The method according to claim 22, wherein the portion of said fresh nutrient in the merging step further comprises a substance, such as chloramphenicol, affecting the growth of said microorganisms.

24. The method according to claim 22 further comprises a step of re-circulating at least one of the newly formed droplets back and forth in a microfluidic channel, to incubate and monitor growth of said microorganisms.

25. The method according to claim 24, wherein the merging step and re-circulating step are repeated in regular time intervals with a period T.

26. The method according to claim 25, wherein the merging step and re-circulating step are repeated together with a regular change of volume of the newly formed droplets.

27. The method according to claim 26, the volume changes are correlated with monitored growth of said microorganisms.

28. The method according to claim 25, wherein the merging step and re-circulating step are repeated together with an irregular change of volume of the newly formed droplets.

29. The method according to claim 28, the volume changes are correlated with monitored growth of said microorganisms.

30. The method according to claim 24, wherein the merging step and re-circulating step are repeated in irregular time intervals.

31. The method according to claim 30, wherein the merging step and re-circulating step are repeated together with a regular change of volume of the newly formed droplets.

32. The method according to claim 31, wherein the volume changes are correlated with monitored growth of said microorganisms.

33. The method according to claim 30, wherein the merging step and re-circulating step are repeated together with an irregular change of volume of the newly formed droplets.

34. The method according to claim 33, wherein the volume changes are correlated with monitored growth of said microorganisms.
